(19) United States
(12) Patent Application Publication
(10) Pub. No.: US 2016/0009656 A1
(43) Pub. Date: Jan. 14, 2016

(54) SUPRAMOLECULAR MATERIALS MADE OF OLIGOAMIDES

(71) Applicants: ARKEMA FRANCE, Colombes (FR); CENTRE NATIONAL DE LA RECHERCHE SCIENTIFIQUE CNRS, Paris Cedex 16 (FR); ECOLE SUPERIEURE DE PHYSIQUE ET DE CHIMIE INDUSTRIELLES DE LA VILLE DE PARIS, Paris 5 (FR)

(72) Inventors: Ludwik LEIBLER, Paris (FR); Francois TOURNILHAC, Paris (FR); Mathieu CAPELOT, Paris (FR); Reda AGNAOU, Rosny sous Bois (FR); Quentin PINEAU, Evreux (FR)

(73) Assignees: ARKEMA FRANCE, Colombes (FR); CENTRE NATIONAL DE LA RECHERCHE SCIENTIFIQUE (CNRS), Paris Cedex 16 (FR); ECOLE SUPÉRIEURE DE PHYSIQUE ET DE CHIMIE INDUSTRIELLES DE LA VILLE DE PARIS(ESPCI), Paris (FR)

(21) Appl. No.: 14/770,282

(22) PCT Filed: Feb. 25, 2014
(86) PCT No.: PCT/FR2014/050398

§ 371 Date: Aug. 25, 2015

(30) Foreign Application Priority Data
Feb. 25, 2013 (FR) 1351643

(51) Int. Cl. C07D 233/36

(52) U.S. Cl. CPC C07D 233/36

(57) ABSTRACT

The present invention relates to novel supramolecular materials made of straight or branched oligoamides, terminated at each of the ends thereof by an associative group including a nitrogen heterocycle supported by a specific sequence. The invention also relates to the method for preparing said materials, as well as to the uses thereof.

### TECHNICAL FIELD

The present invention relates to novel supramolecular materials based on linear or branched oligoamides terminated at each of their ends by an associative group comprising a nitrogenous heterocycle carried by a specific sequence. It also relates to the process for the preparation of these materials and to their uses.

### BACKGROUND OF THE INVENTION

“Supramolecular” materials are materials consisting of molecules held together by noncovalent bonds, such as hydrogen, ionic and/or hydrophobic bonds. One advantage of these materials is that these physical bonds are reversible, in particular under the influence of the temperature or by the action of a selective solvent.

Some of them additionally have elastomeric properties. In contrast to conventional elastomers, these materials have the advantage of being able to fluidify above a certain temperature, which facilitates the processing thereof, in particular the satisfactory filling of molds, and also the recycling thereof. Although they do not consist of crosslinked polymers but of small molecules, these materials are, like elastomers, capable of exhibiting a dimensional stability over very long times and of recovering their initial form after large deformations. They can be used to manufacture thermal or acoustic insulators, cables, sheaths, footwear soles, packagings, elastic clamp collars, vacuum pipes, or pipes for the transportation of fluids.

Supramolecular materials have already been described by the applicant company, in particular in the documents WO 03/059964, WO 2006/016041, WO 2006/087475, WO 2008/029065, WO 2009/141558, WO 2012/156610 and WO 2012/052673 and in the publication by D. Montarnal et al., Journal of the American Chemical Society, vol. 131, No. 23, pp. 7966-7967 (2009).

Among the abovementioned documents, the document WO 2006/016041 describes a polymer modified by grafting AEIO (or UDETA) or a UDETA derivative. In the case where the polymer is a polyamide, in particular a copolyamide of the Platamid® type, UDETA is grafted to its acid ends (which are of different types in the case of Platamid®). The product from this reaction thus consists of a mixture of compounds exhibiting different chain ends. In addition, this grafting has the effect of increasing the viscosity of the polyamide, until a G″ value corresponding to a viscosity of 17 Pa·s is reached. An even higher viscosity (greater than 7000 Pa·s) is observed for the other grafted polymers exemplified in this document. The polymers described in WO 2009/141558 also have an excessively high viscosity (greater than 225 Pa·s in example 1).

In addition, the document WO 2010/031965 describes a process for compatibilizing two polymers by grafting UDETA. Example 1 discloses a material formed by reaction of adipic acid with a polyamide of PA-11 type, followed by the functionalization of the product obtained using UDETA. Here again, this compound exhibits an excessively high viscosity.

Although these materials and more particularly those exhibiting an elastomeric nature provide highly satisfactory properties at ambient temperature, it would be desirable to have available materials exhibiting good mechanical properties in a solid state approaching those of thermoplastic materials, such as a high breaking stress and a high elongation at break and more particularly a breaking stress greater than 3 MPa, preferably than 4 MPa, indeed even than 10 MPa, over a wide temperature range, while retaining a low melt viscosity similar to that of oils (typically less than 10 Pa·s, indeed even than 1 Pa·s). It would thus be possible to improve the processability of these materials, in particular their capability for wetting fibers, making it possible to use them in the manufacture of fibrous compositions, and also their capability for recycling and their durability.

In point of fact, attempts targeted at reducing the viscosity of known supramolecular materials, by decreasing their molecular weight, have systematically resulted in products exhibiting a breaking stress of less than 4 MPa and a few percent only of elongation at break. The inventors have discovered that the two abovementioned requirements, which are nevertheless difficult to reconcile, could be satisfied by a supramolecular material which differs from those known in that it is semicrystalline, with a melting point of greater than or equal to 120° C. and preferably of greater than or equal to 140° C. Entirely surprisingly, this material comprises an associative group of UDETA type which is supposed to increase the viscosity of the polymers to which it is grafted. Without wishing to be committed to this theory, it appears that the crystallites present below the melting temperature of the material act as crosslinking points and make it possible to confer, on material, the desired thermal and mechanical properties. This material can be obtained according to a process which can be easily operated industrially, by polycondensation of a diacid, of a diamine and of a modifying compound carrying an associative group located at the end of a specific sequence, these reactants being used in given stoichiometric ratios. It has been demonstrated that the end associative groups, provided that they are grafted via this specific sequence, confer the required melting point and the required crystallinity on the material, while the central oligoamide chain exhibits a flexibility sufficient to contribute to the desired mechanical properties being obtained. In a preferred form of the invention, the latter more particularly consists of an alternation of rigid blocks (formed by the diacid units) and flexible blocks (formed by the diamine units). In addition, the modifying compound makes it possible to reduce the size of the chains of molecules constituting the material and to thus confer on it a low melt viscosity.

### SUMMARY OF THE INVENTION

A subject matter of the present invention is thus a material comprising linear or branched oligoamides X.Y terminated at more than 90% by number of their ends by one and the same group -M-L₂-CO-L₁-A, where A is an associative group comprising a nitrogenous heterocycle; L₁ is a chemical bond or a spacer arm consisting of a saturated or unsaturated and cyclic or noncyclic hydrocarbon chain optionally interrupted by one or more oxygen and/or nitrogen atoms; L₂ is a saturated or unsaturated and cyclic or noncyclic hydrocarbon chain which includes at least 4 carbon atoms, which is optionally interrupted by one or more oxo groups and which is optionally substituted by one or more —OH groups and/or one or more chlorine atoms; and M is chosen from CO, NH and O groups (where CO denotes the carbonyl group).

Another subject matter of the invention is a process for the preparation of this material, comprising a stage of polycondensation:
(a) of at least one dicarboxylic acid,
(b) of at least one diamine, and
(c) of at least one modifying compound of formula A-L₁-CO-L₂-W, where A is an associative group comprising a nitrogenous heterocycle; L₁ is a chemical bond or a spacer arm consisting of a saturated or unsaturated and cyclic or noncyclic hydrocarbon chain optionally interrupted by one or more oxygen and/or nitrogen atoms; L₂ is a saturated or unsaturated and cyclic or noncyclic hydrocarbon chain which includes at least 4 carbon atoms, which is optionally interrupted by one or more oxo groups and which is optionally substituted by one or more —OH groups and/or one or more chlorine atoms; and W is a reactive group capable of reacting:
   either with the amine functional groups of the diamine, the molar ratio of the acid functional groups of the diacid to the reactive functional groups of the modifying compound in this case ranging from 1 to 8, it being understood that the number of moles of amine functional groups of the diamine is equal to the sum of the number of moles of the abovementioned acid and reactive functional groups,
   or with the acid functional groups of the diacid, the molar ratio of the amine functional groups of the diamine to the reactive functional groups of the modifying compound in this case ranging from 1 to 8, it being understood that the number of moles of acid functional groups of the diacid is equal to the sum of the number of moles of the abovementioned amine and reactive functional groups.

A further subject matter of the present invention is the uses of this material as additive in asphalts, as matrix for composite materials, as hot-melt adhesive or as additive in hot-melt adhesives.

Another subject matter of the present invention is the compound of formula:

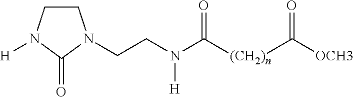

where n ranges from 4 to 14, and also its use as modifying compound in the process according to the invention.

### DETAILED DESCRIPTION

As a preliminary, it should be noted that the expression “of between” should be interpreted in the present description as including the limits cited.

As indicated above, the supramolecular material according to the invention comprises linear or branched oligoamides X.Y terminated at more than 90% by number of their ends by one and the same specific unit and preferably at 100%.

The term oligoamides denotes polycondensates having a low number-average molecular weight Mn. The value of Mn can be predicted, as a function of the molar ratios of the reactants involved in the polycondensation and of the degree of progression of the reaction, by using conventional formulae known to a person skilled in the art. The molar ratios will be chosen in such a way that the Mn predicted by the formula of Stockmayer, W. H. (Journal of Polymer Science, 1952, 9, 67-71), and assuming a complete conversion, is less than 10 000 g/mol and preferably less than 4500 g/mol.

“Oligoamides X.Y” is understood to mean homopolyamides or copolyamides of low mass obtained from at least one diacid and at least one diamine, in contrast to the oligoamides comprising units obtained by condensation of amino acids.

The term “linear oligoamides” denotes oligoamides comprising, in the chain, only diacid and diamine units; the term “branched oligoamides” denotes oligoamides comprising, in the chain, one or more polyacid or polyamine units with a functionality greater than 2.

In addition, the expression “more than 90% by number . . . ” is understood to mean that less than 10% by number of the ends of the molecules constituting the material can optionally result from an incomplete conversion of the reaction or can originate from monofunctional entities present in the monomers used. More particularly, acid, amine or alkyl functionalities are concerned, which functionalities can be observed by conventional analytical techniques, such as potentiometry, proton NMR spectroscopy or infrared spectroscopy.

According to a first embodiment of the invention, this material comprises oligomers corresponding to the formula (Ia) below:

X—NH—Ra—NH—[CO—Rb—CO—NH—Ra—NH—]ₐ—X (Ia)

where:

Ra denotes a saturated or unsaturated hydrocarbon chain optionally interrupted by one or more oxygen and/or nitrogen atoms,

Rb denotes a saturated or unsaturated hydrocarbon chain,

a denotes the mean number of units per chain and is greater than 0 and less than or equal to 20, preferably less than or equal to 9 and more preferably still of between 1 and 3,

X denotes an A-L₁-CO-L₂-CO— group

where:
A is an associative group comprising a nitrogenous heterocycle,
L₁ is a chemical bond or a spacer arm consisting of a saturated or unsaturated and cyclic or noncyclic hydrocarbon chain optionally interrupted by one or more nitrogen and/or oxygen atoms,
L₂ is a saturated or unsaturated and cyclic or noncyclic hydrocarbon chain which includes at least 4 carbon atoms, which is optionally interrupted by one or more oxo groups and which is optionally substituted by one or more —OH groups and/or one or more chlorine atoms.
In the formula (Ia) above, it is preferable for the X group to comprise an L_{1′}-CO-L₂-CO— sequence where L₂ denotes a linear alkylene chain and L_{1′} denotes an —NH—CH₂—CH₂— chain.

According to a second embodiment, the material according to the invention comprises oligomers corresponding to the formula (Ib) below:

X′—CO—Rb—CO—[NH—Ra—NH—CO—Rb—CO—]ₐ—X′ (Ib)

where:

Ra, Rb and a have the definitions indicated above,

X′ denotes an A-L₁-CO-L₂-M₁- group

where:
A, L₁ and L₂ have the definitions indicated above,
M₁ is an oxygen atom or an NH group.

In the formulae (Ia) and (Ib) above, it is preferable for one at least of the Ra and Rb chains not to consist of a linear alkylene chain.

The reactants used in the process for the synthesis of these materials will now be described in more detail.

Dicarboxylic Acid

The dicarboxylic acid employed in the first stage of the process according to the invention advantageously comprises from 4 to 100 carbon atoms. It can be a saturated linear dicarboxylic acid including from 4 to 22 carbon atoms, such as succinic acid, glutaric acid, adipic acid, pimelic acid, suberic acid, azaleic acid, sebacic acid, undecanedioic acid, dodecanedioic acid, brassylic acid, tetradecanedioic acid, pentadecanedioic acid, thapsic acid, octadecanedioic acid and their mixtures. In an alternative form, the dicarboxylic acid used in this invention can be a saturated branched dicarboxylic acid including, for example, from 6 to 10 carbon atoms, such as 3,3-dimethylglutaric acid.

In another alternative form, it can be an aromatic diacid, such as terephthalic acid, isophthalic acid, naphthalenic diacids and their mixtures. In another alternative form, the diacid can be cycloaliphatic. In the latter case, it can comprise the following carbon-based backbones: norbornylmethane, cyclohexylmethane, dicyclohexylmethane, dicyclohexylpropane, di(methylcyclohexyl) or di(methylcyclohexyl)propane.

Use may also be made, according to the invention, of the dimers of carboxylic acids of vegetable origin, consisting of two monomers of fatty acids which are identical or different, optionally as a mixture with monomers and/or trimers of fatty acids, these mixtures being chosen so that the polycondensate obtained remains below the chemical gel point. These compounds of vegetable origin may or may not be unsaturated. They result from the oligomerization (in particular from the dimerization) of unsaturated fatty acids, such as undecylenic acid, myristoleic acid, palmitoleic acid, oleic acid, linoleic acid, linolenic acid, ricinoleic acid, eicosenoic acid and docosenoic acid, which are normally found in pine oil (tall oil fatty acids), rapeseed oil, corn oil, sunflower oil, soybean oil, grape seed oil, linseed oil and jojoba oil, and also eicosapentanoic acid and docosahexanoic acid, which are found in fish oils.

Use may thus be made of a mixture of oligomers of fatty acids comprising at least 30% by weight, indeed even at least 50% by weight and preferably at least 70% by weight of dimers of linear or cyclic C₁₈ fatty acids which are optionally partially or completely hydrogenated, said mixture comprising a low percentage of monomers of fatty acids (typically less than 5%, preferably less than 2% and more preferably less than 1% by weight in total) and preferably including different isomers of one and the same fatty acid dimer.

Mention may be made, as examples of mixtures comprising dimers of fatty acids, of those sold by Croda under the trade names Pripol® 1006, 1009, 1012, 1013, 1017, 1022, 1025 and 1027, by Arizona Chemicals under the trade name Unidyme® 14, by BASF under the trade name Empol® 1008, 1016 or 1018, or by Oleon under the trade name Radiacid® 0980.

Use may also be made, according to the invention, of the diacids and polyacids derived from fatty substances as described in the document FR 2 962 131 A1 and represented in figures (IIa), (IIb) and (IIc) of said document. These diacids are obtained by modification of fatty acids or of mixtures of fatty acids of natural origin (rapeseed oil, oleic acid, for example) using thiols carrying an acid functional group, by thiol-ene chemistry.

Use is preferably made, in the present invention, of an aromatic diacid, such as terephthalic acid, or a saturated linear dicarboxylic acid including from 4 to 12 carbon atoms, such as adipic acid or sebacic acid.

Diamine

The diamine can be chosen from any saturated or unsaturated and linear, branched or cyclic compound carrying two primary amine functional groups. In the present description, the term “diamine” thus encompasses in particular polyamines comprising only two primary amine functional groups and at least one other secondary or tertiary amine functional group.

The diamine can thus be a compound of formula (II):

H₂N—(CH₂)ₚ—NH₂ (II)

where p is an integer ranging from 3 to 20, such as cadaverine, putrescine, hexamethylenediamine or 1,12-diaminododecane.

In an alternative form, use may be made of cycloaliphatic diamines and diamines comprising a branched chain, such as isophoronediamine or bis(3-methyl-4-aminocyclochexyl)methane (BMACM), or also dimer diamines resulting from vegetable fatty acids, in particular from C₁₈ fatty acids, which may be partially or completely hydrogenated, such as those mentioned above. These dimer diamines are available in particular from Croda, for example under the trade name Priamine® 1074 or 1073.

Other examples of diamines are linear diamines comprising heteroatoms (N) in their chain, in particular the compounds of formula (III):

H₂N—(CHR₁)ₘ—[NH—(CH₂)ₓ]_{y}—NH—(CHR₂)ₙ—NH₂ (III)

in which:

R₁ and R₂ independently denote a hydrogen atom or a C₁-C₆ alkyl group, such as a methyl group,

m and n independently denote an integer ranging from 1 to 3,

x denotes an integer ranging from 1 to 6,

y denotes an integer ranging from 0 to 2.

In the formula (III) above, at least one and preferably all of the conditions below are satisfied:
R₁ and R₂ denote a hydrogen atom,
m+n is equal to 2, 3 or 6, preferably to 2,
x denotes an integer ranging from 2 to 4,
y is equal to 0 or 1, preferably to 0.

Preferred examples of polyamines of formula (III) are DETA (diethylenetriamine), TETA (triethylenetetramine), TEPA (tetraethylenepentamine), spermine and dihexylenetriamine.

Yet another type of diamines, which comprise heteroatoms (O) in their chain and which can be used according to the invention, consist of polyetheramines consisting in particular of a linear or branched polyether chain, such as a polypropylene glycol, polyethylene glycol, or polytetramethylene glycol chain, and their copolymers, each end of which carries a primary amine group. Such compounds are available in particular from Huntsman under the trade names Jeffamine® D, ED or EDR series.

Use may also be made, according to the invention, of diamines and polyamines which are both branched and carry a heteroatom (S) in their chain, such as those described in the document FR 2 962 131 A1 and represented in figures (IIa), (IIb) and (IIc) of said document. These diamines are obtained in particular by modification of unsaturated triglycerides of natural origin (for example rapeseed oil) using thiols carrying an amine functional group (such as cysteamine), by thiol-ene chemistry.

In an alternative form, linear diamines, linear diamines comprising heteroatoms (O, S or N) in their chain, branched-chain diamines and cycloaliphatic diamines will be able to be used as mixtures in which, preferably, the molar fraction of linear diamines not comprising a heteroatom in their chain does not exceed 500 of the total number of moles of diamines involved.

As in the case of the diacid, the diamine according to the invention can optionally be used as a mixture with monoamines or polyamines, these mixtures being chosen so that the polycondensate obtained remains below the chemical gel point.

It is preferable, according to the invention, to use diamines comprising a flexible sequence supposed to be not very crystallizable. These amines can be selected from the following list: polyetheramines comprising a linear or branched polyether chain (in particular of poly(ethylene glycol), poly(propylene glycol) or poly(tetramethylene glycol) structure and their copolymers), each end of which carries a primary amine group, branched diamines and polyamines, the chain of which comprises a sulfur atom, and dimer diamines resulting from vegetable fatty acids optionally partially or completely hydrogenated, as described above.

Dimer diamines resulting from vegetable fatty acids optionally partially or completely hydrogenated are the preferred diamines.

Modifying Compound

In the process according to the invention, the diamine or dicarboxylic acid reacts with a modifying compound carrying, on the one hand, an associative group comprising a nitrogenous heterocycle and, on the other hand, a reactive functional group capable of reacting with the diamine or the diacid respectively.

This modifying compound has the general formula: A-L₁-CO-L₂-W, where A is an associative group comprising a nitrogenous heterocycle; L₁ is a chemical bond or a spacer arm consisting of a saturated or unsaturated and cyclic or noncyclic hydrocarbon chain optionally interrupted by one or more oxygen and/or nitrogen atoms; L₂ is a saturated or unsaturated and cyclic or noncyclic hydrocarbon chain which includes at least 4 carbon atoms, which is optionally interrupted by one or more oxo groups and which is optionally substituted by one or more —OH groups and/or one or more chlorine atoms; and W is a reactive group capable of reacting with amine or acid functional groups of the diamine or of the diacid respectively.

Specific associative groups are as follows:

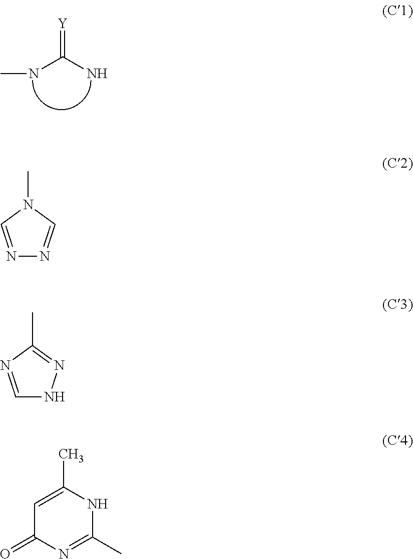

where:

Y is chosen from an oxygen or sulfur atom or an NH group, and

the bond represented by a circle arc in C′l is chosen from —CH₂—CH₂—, —CH═CH— and —NH—CH₂—.

Mention may thus be made, as examples of associative groups, of the imidazolidonyl, triazolyl, triazinyl, bis-ureyl and ureido-pyrimidyl groups. An associative group preferred for use in the present invention is the imidazolidonyl group.

In a preferred embodiment of the invention, the modifying compound reacts with the amine functional groups of the diamine. In this case, the molar ratio of the acid functional groups of the diacid to the reactive functional groups of the modifying compound is of between 1 and 8, for example between 1 and 3, it being understood that the number of moles of amine functional groups of the diamine is equal to the sum of the number of moles of the abovementioned acid and reactive functional groups.

Examples of such modifying compounds are those of formula A-L₁-CO-L₂-W₁

where:
A, L₁ and L₂ have the meanings indicated above, and
W₁ is a carboxylic acid, ester, anhydride or acyl chloride functional group.

According to a preferred embodiment of the invention, the modifying compound corresponds to the formula (IV):

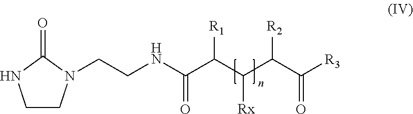

in which R₁, R₂ and each of the Rx groups independently denote a hydrogen atom, an —OH group or a —CH₃ group, preferably a hydrogen atom; n ranges from 2 to 12 and is preferably equal to 2, 4 or 6, more preferably to 2; and R₃ denotes an —OH group, an —OCH₃ group or a chlorine atom, preferably an —OCH₃ group.

More preferably still, the modifying compound corresponds to the following formula (V):

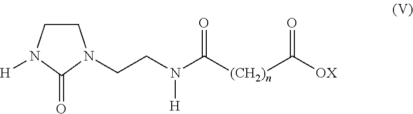

where n is of between 4 and 14 and X═H or CH₃.

This compound is novel and thus also forms the subject matter of this invention.

The abovementioned modifying compounds can be obtained by reacting UDETA (or 1-(2-aminoethyl)imidazolidin-2-one) with:
an alicyclic anhydride, such as glutaric anhydride, succinic anhydride, maleic anhydride, citraconic anhydride, diglycolic anhydride or itaconic anhydride, or
an aromatic anhydride, such as 1,8-naphthalic anhydride, phthalic anhydride and isatoic anhydride, or
a dicarboxylic acid, such as adipic acid, or
a dicarboxylic acid ester, such as dimethyl adipate or dimethyl terephthalate, or
a dicarboxylic acid chloride.

UDETA can itself be prepared by reaction of urea with diethylenetriamine (DETA). Other similar modifying compounds can be obtained by replacing UDETA with UTETA or UTEPA, which can respectively be prepared by reacting urea with triethylenetetramine (TETA) and tetraethylenepentamine (TEPA).

According to another embodiment of the invention, the modifying compound is chosen from the compounds corresponding to the formula (VI):

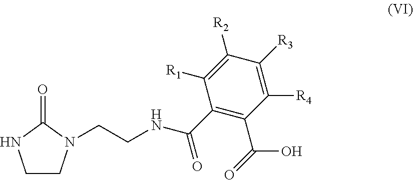

in which R₁, R₂, R₃ and R₄ independently denote a hydrogen atom, a hydroxyl group, an amino group, a nitro group or an alkyl group, and also their positional isomers and their esters, acyl chlorides and anhydrides.

As indicated above, the modifying compound can, in an alternative form, react with the acid functional groups of the dicarboxylic acid used in the process according to the invention. In this case, the molar ratio of the amine functional groups of the diamine to the reactive functional groups of the modifying compound is of between 1 and 8, it being understood that the number of moles of acid functional groups of the diacid is equal to the sum of the number of moles of the abovementioned amine and reactive functional groups.

In this embodiment, the modifying compound generally has the formula: A-L₁-CO-L₂-W₂

where:
A, L₁ and L₂ have the meanings indicated above, and
W₂ is an OH or NH₂ group.

An example of such a modifying compound corresponds to the formula (VII):

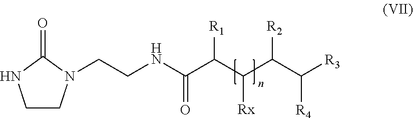

in which R₁, R₂, Rx and n have the meanings given above, R₃ denotes an OH or NH₂ group and R₄ denotes a hydrogen atom or an alkyl group.

Another modifying compound which can be used in this embodiment is the following ureido-pyrimidyl (UPy) derivative:

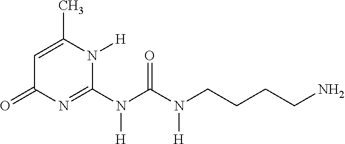 [SMILES: Cc1cc(=O)nc(NC(=O)NCCCCN)[nH]1]

the synthesis of which is described in example 1 of the document FR 2 954 941.

As indicated above, in the two embodiments of the invention mentioned above, that is to say (i) when the modifying compound reacts with the amine functional groups of the diamine and (ii) when the modifying compound reacts with the acid functional groups of the diacid, the diacid can be replaced by a mixture of monoacid B1, diacid B2, triacid B3, and the like, and/or the diamine can be replaced by a mixture of monoamine A1, diamine A2, triamine A3, and the like. In these cases, the amounts of polyacids and polyamines with a functionality of greater than or equal to have to be limited so as not to reach the chemical gel point.

For this, the numbers of moles of each component of the mixtures are chosen so that the following product P remains strictly less than 1:

$$P = ( \frac{{\sum }_{i} {n}_{{A}_{i}} {i}^{2}}{{\sum }_{i} {n}_{{A}_{i}} i} - 1 ) \cdot ( \frac{{\sum }_{j} {n}_{{B}_{i}} {j}^{2}}{{\sum }_{j} {n}_{{B}_{j}} j} - 1 )$$

where:

n_{A1} denotes, in the case (i), the number of moles of monoamine and, in the case (ii), the sum of the numbers of moles of monoamine and of modifying compound,

n_{B1} denotes, in the case (i), the sum of the numbers of moles of monoacid and of modifying compound and, in the case (ii), the number of moles of monoacid,

n_{A2} denotes the number of moles of diamine,

n_{B2} denotes the number of moles of diacid,

n_{A3} denotes the number of moles of triamine,

n_{B3} denotes the number of moles of triacid,

n_{Ai} (i>3) denotes the number of moles of polyamine comprising i amine functional groups,

n_{Bj} (j>3) denotes the number of moles of polyacid comprising j acid functional groups.

Synthesis of the Supramolecular Material

In the process according to the invention, use is made of a modifying compound which has been synthesized, isolated and optionally purified beforehand, so as to exhibit a purity of at least 90%. The synthesis of this compound can optionally constitute a preliminary stage of the process according to the invention. On the other hand, the synthesis of this compound in situ is excluded in order to avoid the formation of undesirable products, such as that illustrated below, obtained from UDETA and a linear dicarboxylic acid:

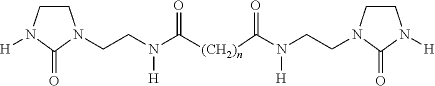

The dicarboxylic acid, the diamine and the modifying compound are introduced, in the molar ratios indicated above, either simultaneously or successively in any order, into a reactor. The polycondensation reaction is carried out at a temperature of 50 to 200° C., for example of 160 to 200° C., for a period of time ranging from 1 to 24 hours, in particular from 4 to 6 h. The reaction is normally carried out with stirring, for example at a speed of from 200 to 350 revolutions/min. It is preferably carried out in the absence of solvent and advantageously under a stream of inert gas, such as nitrogen, which makes it possible to discharge the water and the methanol which are produced during the reaction and to thus shift the reaction towards the formation of the desired oligoamides.

The progression of the reaction can be monitored by infrared spectroscopy. The disappearance of the bands characteristic of the diacid or of the diamine in favor of the amide makes it possible to determine the time when the reaction is complete.

This reaction produces oligoamides of variable chain lengths, functionalized at each of their ends by associative units and thus capable of combining with one another via hydrogen bonds which are reversible as a function of the temperature. It has been observed that the length of the chains which are obtained, and also their crystallizable fraction, is directly related to the abovementioned stoichiometric ratios. The latter are thus adjusted in order to make it possible to obtain a material having the crystallinity required in order to confer on it good mechanical properties and a molecular weight which is sufficiently low to exhibit a low melt viscosity.

The polycondensate obtained on conclusion of this process is semicrystalline, characterized, for example, by an enthalpy of fusion of greater than 10 J/g, and it generally has a melting temperature (T_{f}) of between 120 and 260° C., preferably between 130 and 180° C. and more preferably between 140 and 170° C., and a glass transition temperature (T_{g}) generally of between −25° C. and 100° C. and preferably between −25° C. and 10° C.

Its number-average molecular weight, as measured by GPC, is generally less than 4500 g/mol, for example of between 1000 and 3000 g/mol, but it can, in an alternative form, range up to 10 000 g/mol.

This material exhibits a low melt viscosity, generally of less than 10 Pa·s, preferably of less than 1 Pa·s and typically of between 0.1 and 0.5 Pa·s, at 30° C. above its melting point, and good mechanical properties at ambient temperature and in a broad temperature range above ambient temperature, which are reflected by a breaking stress greater than 1 MPa, in particular greater than 3 MPa, preferably greater than 4 MPa, indeed even greater than 10 MPa, and optionally a ductile tensile behavior.

The abovementioned properties are measured according to the techniques given in the Examples part of this description. The material according to the invention exhibits at least one and preferably all of these properties.

The material according to the invention can particularly be used to manufacture leaktight seals, thermal or acoustic insulators, footwear soles, packagings, coatings (paints, films, cosmetic products), systems for trapping and releasing active principles, vacuum pipes and generally parts which have to exhibit good tear and/or fatigue strengths, rheological additives, additives for asphalt or additives for hot-melt adhesives, or also matrices of composites.

A better understanding of the invention will be obtained in the light of the following examples, given for purposes of illustration only and which do not have the aim of restrictiong the scope of the invention, defined by the appended claims.

### EXAMPLES

Measurement methods
1. Thermal analysis: the samples were characterized by differential scanning calorimetry (DSC). The following protocol was applied: first heating at 10° C./min from −100° C. to 250° C., isotherm of 5 min at 250° C., cooling at −10° C./min down to −100° C., isotherm at −100° C. for 5 min and then second heating up to 250° C. at 10° C./min. The enthalpies of fusion were measured using the TA Universal Analysis software in “Sigmoidal tangent” mode. The melting temperatures were determined at the tip of the peak and the glass transition temperatures at the inflection point.
2. Rheology: the viscosity of the samples was measured with a shear rate of 100 s⁻¹, using a rheometer equipped with a cone-plate geometry with a diameter of 50 mm. The sample was placed under the geometry preheated to 180° C.
3. Mechanical properties: the uniaxial tensile properties were studied on samples in the form of 12 mm×2 mm×1.2 mm dumbbells using an Instron 5564 tensile testing device. The tests were carried out at ambient temperature, with a rate of 2 mm/min. Three independent tests were carried out for each material and the mean of the results of these three tests was subsequently calculated.

### Example 1

### Preparation of Modifying Compounds according to the Invention

This example illustrates the synthesis of different modifying compounds used according to the invention.

### Example 1A

### Anhydride Opening

The reaction scheme was as follows:

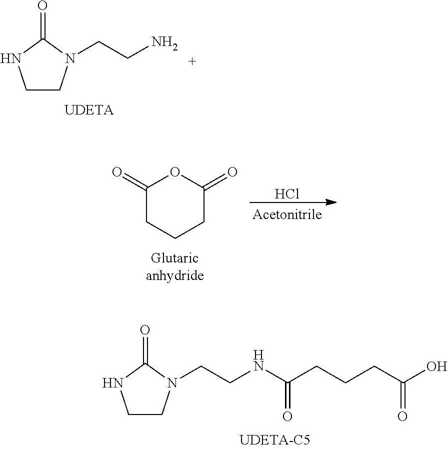 [SMILES: NCCN1CCNC1=O ; O=C1CCCC(=O)O1 ; Cl ; O=C(O)CCCC(=O)NCCN1CCNC1=O]

In a first step, the glutaric anhydride (7.95 g, i.e. 0.07 mol) was introduced into a dropping funnel. The acetonitrile (15 ml) was added to the anhydride and dissolved at 60° C. with stirring (the dissolution was not complete). A solution of UDETA (10 g, i.e. 0.077 mol) in acetonitrile (15 ml) was prepared and introduced into a two-necked round-bottomed flask provided with a magnetic bar and surmounted by the dropping funnel. The round-bottomed flask was introduced into a water bath at ambient temperature, a few drops of 36-38% hydrochloric acid (i.e., 0.001 mol) were subsequently added and the anhydride solution was introduced dropwise over a period of 30 minutes. The reaction mixture was kept stirred at ambient temperature for 20 h and then at 40° C. for 4 hours. The product, which precipitated in the form of a white powder as it was formed, was recovered by filtration (vacuum pump), washed twice with acetonitrile and dried under a bell jar at ambient temperature. The product obtained was a white powder characterized chemically: the purity of the final product could be easily determined by proton NMR spectroscopy.

This process can be applied to the synthesis of similar modifying compounds having different chain sizes.

### Example 1B

### Condensation of UDETA with a Diester

The reaction scheme was as follows:

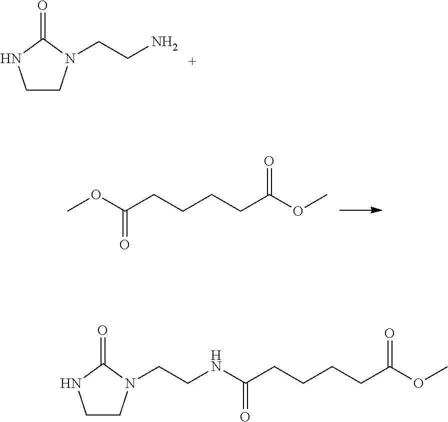

The UDETA (30 g, i.e. 0.232 mol) and the dimethyl adipate (364.13 g, i.e. 2.090 mol) in large excess (9 equivalents) were introduced into a two-necked round-bottomed flask provided with a magnetic bar. The transparent starting mixture was stirred and a stream of nitrogen was deployed in order to remove the methanol which was formed in the reaction medium. The round-bottomed flask was placed in a bath of silicone oil heated at 140° C. for a period of 6 hours. At the end of the reaction, the excess diester was removed by distillation at 160° C. under static vacuum at the start, at 180° C. under static vacuum when the distillation slowed down and then under dynamic vacuum at 160° C. in order to recover the greatest possible amount of diester. Finally, the product formed was washed five times with pentane and dried under a bell jar at ambient temperature for 24 hours. The final product (UDETA-C6) could be characterized by NMR spectroscopy and exhibited a good purity.

A similar process can be carried out while replacing the diester used above with a dicarboxylic acid or a dicarboxylic acid chloride.

### Example 1C

### Lactone Opening

The reaction scheme was as follows:

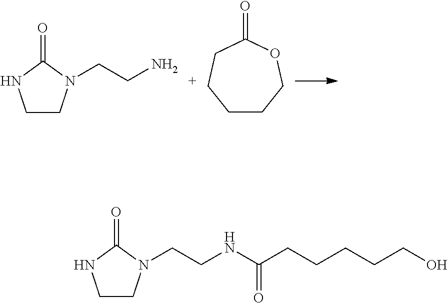

In a first step, the UDETA (20 g, i.e. 0.155 mol) was dissolved at ambient temperature in 30 ml of acetonitrile in a two-necked round-bottomed flask provided with a magnetic bar. The round-bottomed flask was surmounted by a dropping funnel into which a solution of 18.3 ml (0.17 mol) of caprolactone in 15 ml of acetonitrile has been introduced. This solution was added dropwise at ambient temperature to the reaction mixture over a period of 30 minutes. The mixture was subsequently left stirring for 12 hours and then at 40° C. for an additional 4 hours. The solution was concentrated on a rotary evaporator and placed in a freezer for 12 hours in order to crystallize the reaction product, which was subsequently recovered by filtration, washed with acetonitrile and dried under a bell jar for 6 hours. The reaction product could be easily analyzed by proton NMR spectroscopy in order to determine the purity thereof.

A great variety of lactones can be opened in the same way and can give rise to functionalized carbon-based chains.

### Example 2

### Preparation of Supramolecular Oligoamide Based on Acid Dimer

An unsaturated fatty diacid (Pripol® 1009), a diamine comprising a ring and an unsaturation (Priamine® 1074) and a modifying compound obtained as described in example 1B (UDETA-C6) were condensed in order to obtain the compounds of the following formula:

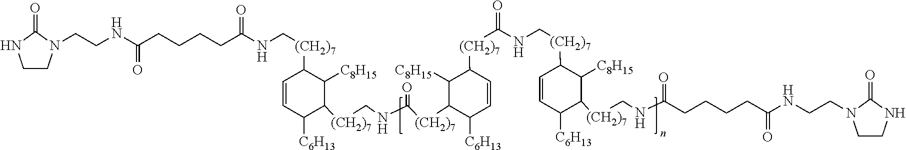

where n varied from 0 to 3.

The reaction was carried out in a jacketed reactor with a diameter of 60 mm and with a nominal volume of 1 l, regulated using a thermostatically controlled bath having circulation of silicone oil, the pipes of which were reinforced by a metal sheath. This reactor was surmounted by a mechanical stirrer and was provided with a bottom valve, with a gas inlet and with a bubbler. The reaction was monitored by infrared spectroscopy and the reaction products were analyzed by NMR spectroscopy and GPC.

The product corresponding to n=1 was thus synthesized in the following way.

52.5 g (0.18 mol) of Pripol© 1009 were weighed and introduced into the reactor. 101.01 g (0.37 mol) of Priamine® 1074 and then 50 g (i.e., 0.18 mol) of UDETA-C6 were subsequently introduced into the reactor. The reactor was then closed and heated to 180° C. while stirring at 280 revolutions/minute. A stream of nitrogen of 300 ml/minute was deployed using a pipe which can withstand high temperatures. This pipe was introduced as close as possible to the stirred mixture. A gas outlet connected to a bubbler made it possible to confirm the leaktightness of the reactor and to retain the methanol formed during the reaction. Monitoring by infrared (IR) spectroscopy was deployed in order to determine the moment when the heating had to be halted. Analysis by IR spectroscopy confirmed the disappearance of the ν(C═O) band of the carboxylate functional group at approximately 1394 cm⁻¹ and the appearance of the ν(C═O) band of the amide at approximately 1650 cm⁻¹. The reaction was complete after 6 hours and the oligomer obtained was recovered at the bottom valve into a Teflon beaker. The product obtained crystallized fairly rapidly in the beaker and did not adhere thereto. It could thus be easily recovered and analyzed.

All the samples of this example were obtained in the same way. The products differed in their chain sizes, which themselves depended on the value of n, that is to say on the molar ratio of the acid functional groups of the diacid to the reactive functional groups of the modifying compound. In addition, two comparative examples were carried out, using modifying compounds which do not come within the scope of the present invention. Finally, an additional example was carried out while substituting Pripol® 1017 for Pripol® 1009. The various tests carried out are collated in the table below.

| n = 0 | M (g/mol) | n (mol) | w (g) | Mn (g/mol) |
|---|---|---|---|---|
| Pripol 1009 | 285 | 0 | 0.0 | 1026 |
| Priamine | 274 | 0.184325 | 50.5 | |
| UDETA-C6 | 271.26 | 0.184325 | 50 | |

| n = 1 | M (g/mol) | n (mol) | w (g) | Mn (g/mol) |
|---|---|---|---|---|
| Pripol 1009 | 285 | 0.184325 | 52.5 | 2108 |
| Priamine | 274 | 0.36865 | 101.0 | |
| UDETA-C6 | 271.26 | 0.184325 | 50 | |

| n = 2 | M (g/mol) | n (mol) | w (g) | Mn (g/mol) |
|---|---|---|---|---|
| Pripol 1009 | 285 | 0.03985965 | 11.4 | 3190 |
| Priamine | 274 | 0.05978947 | 16.4 | |
| UDETA-C6 | 271.26 | 0.01992982 | 5.40616421 | |

| n = 3 | M (g/mol) | n (mol) | w (g) | Mn (g/mol) |
|---|---|---|---|---|
| Pripol 1009 | 285 | 0.04568421 | 13.0 | 4272 |
| Priamine | 274 | 0.06091228 | 16.7 | |
| UDETA-C6 | 271.26 | 0.01522807 | 4.2 | |

| Counter- | | | | |
|---|---|---|---|---|
| example 2-1 | M (g/mol) | n (mol) | w (g) | Mn (g/mol) |
| Pripol 1009 | 285 | 0.00839416 | 2.41 | 1911 |
| Priamine | 274 | 0.01678832 | 4.60 | |
| Monolauroyl | 327.51 | 0.00839416 | 2.75 | |
| adipamide* | | | | |

*product of the reaction of dodecylamine with dimethyl adipate

| Counter- | | | | |
|---|---|---|---|---|
| example 2-2 | M (g/mol) | n (mol) | w (g) | Mn (g/mol) |
| Pripol 1009 | 285 | 0.00930657 | 2.46 | 1656 |
| Priamine | 274 | 0.01861314 | 5.10 | |
| Laurie acid | 200.32 | 0.00930657 | 1.86 | |
| Pripol 1017 | 288.66 | 0.02764498 | 7.98 | 2080 |
| Priamine | 274 | 0.20189781 | 55.32 | |
| UDETA-C6 | 271.26 | 0.02764875 | 7.50 | |

### Example 3

### Preparation of a Supramolecular Oligoamide Based on Adipic Acid

A second series of samples was prepared in a similar way to example 2, Pripol® 1009 being replaced with adipic acid, in order to obtain materials corresponding to the following formula:

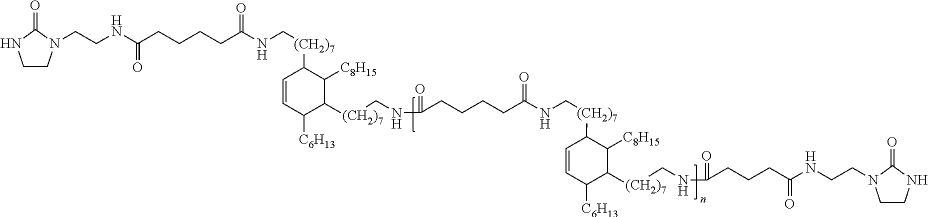

where n varied from 0 to 3.

As in example 2, a comparative sample was also prepared by replacing UDETA-C6 with a modifying compound not coming within the scope of the invention. The conditions for the preparation of the samples of this series are summarized in the table below:

| n = 0 | M (g/mol) | n (mol) | w (g) | Mn (g/mol) |
|---|---|---|---|---|
| Adipic acid | 72.06 | 0 | 0.00 | 1026 |
| Priamine | 274 | 0.184325 | 50.50 | |
| UDETA-C6 | 271.26 | 0.184325 | 50.00 | |

| n = 0.33 | M (g/mol) | n (mol) | w (g) | Mn (g/mol) |
|---|---|---|---|---|
| Adipic acid | 72.06 | 0.04204 | 3.03 | 1387 |
| Priamine | 274 | 0.16819 | 46.11 | |
| UDETA-C6 | 271.26 | | | |

| n = 1 | M (g/mol) | n (mol) | w (g) | Mn (g/mol) |
|---|---|---|---|---|
| Adipic acid | 72.06 | 0.04239475 | 3.09 | 1062 |
| Priamine | 274 | 0.0847895 | 23.27 | |
| UDETA-C6 | 271.26 | 0.04239475 | 11.50 | |

| n = 2 | M (g/mol) | n (mol) | w (g) | Mn (g/mol) |
|---|---|---|---|---|
| Adipic acid | 72.06 | 0.03880779 | 2.80 | 1372 |
| Priamine | 274 | 0.05821168 | 15.95 | |
| UDETA-C6 | 271.26 | 0.01940389 | 5.26 | |

| n = 3 | M (g/mol) | n (mol) | w (g) | Mn (g/mol) |
|---|---|---|---|---|
| Adipic acid | 72.06 | 0.04125 | 3.00 | 1693 |
| Priamine | 274 | 0.055 | 15.07 | |
| UDETA-C6 | 271.26 | 0.01375 | 3.80 | |

| Counter- | | | | |
|---|---|---|---|---|
| example 3-1 | M (g/mol) | n (mol) | w (g) | Mn (g/mol) |
| Adipic acid | 72.06 | 0.03880779 | 2.82 | 1201 |
| Priamine | 274 | 0.05821168 | 15.95 | |
| Monolauroyl | 185.35 | 0.01940389 | 3.60 | |
| adipamide* | | | | |

*product of the reaction of dodecylamine with dimethyl adipate

### Example 4

### Evaluation of the Properties of the Supramolecular Materials

The materials prepared in examples 2 and 3 were subjected to the various tests presented above, for the purpose of evaluating their physicochemical and mechanical properties. The materials synthesized according to example 3 were annealed at 100° C. for 1 h before measuring their enthalpy of fusion.

The results of these tests are collated in the table below:

| | | Mn, | | | η | Breaking | Elongation | ΔH |
|---|---|---|---|---|---|---|---|---|
| | | theo. | T_{g} | Tₘ | (180° C., | stress | at break | fus. |
| | n | (g/mol) | (° C.) | (° C.) | 100 s⁻¹) | (MPa) | (%) | (J/g) |
| Ex. 2 | | | | | | | | |
| Counterex. | 0 | 1026 | 1.3 | 164 | 0.08 | / | / | 41 |
| 2-1 | 0.33 | 1387 | 3.2 | 163 | 0.12 | / | / | 26 |
| | 1 | 2108 | −9.3 | 160 | 0.16 | 4.6 | 30 | 25 |
| | 2 | 3190 | −16.6 | 155.6 | 0.25 | 1.8 | 36 | 15 |
| | 3 | 4272 | −18 | 153.2 | 0.39 | 1.3 | 31 | 6.2 |
| | 1 | 3303 | −24.7 | 107 | 0.08 | 2.3 | 15 | 6.2 |
| Counterex. | 1 | 3048 | / | 21 | 0.08 | Viscous | / | / |
| 2-2 | | | | | | liquid | | |
| Ex. 3 | | | | | | | | |
| Counterex. | 0 | 1026 | −3.3 | 166 | 0.08 | / | / | 41 |
| 3-1 | 1 | 1683 | 6.1 | 155.8 | | 7 | 7.7 | 27.08 |
| | 2 | 2339 | 9 | 156 | 0.21 | 7.6 | 24.7 | 21 |
| | 3 | 3005 | −4.6 | 149 | | 12.2 | 28 | 12.37 |
| | 1 | 1403 | −26.2 | 111.9 | 0.19 | 3.8 | 8 | 22 |

It emerges from these tests that the replacement of the modifying compound according to the invention by another compound not comprising a nitrogenous heterocycle results in a material having a melting point lower than 120° C., which is insufficient for numerous applications, indeed even in a material which is not solid but exists in the form of a viscous liquid at ambient temperature. In addition, the materials prepared in the absence of diacid (n=0) or in the presence of an insufficient amount of diacid (n=0.33) were too brittle to remove from the mold and thus could not be tested.

In addition, it has been observed that the above properties of the materials according to the invention were similar to those obtained by using a typical polyamide-6, with a molecular weight equal to 18 000 g/mol. However, their melt viscosity is more than 100 times lower than that of polymers of this type, which greatly facilitates their use.

### Example 5

### (Comparative): Materials Obtained by Replacing the Modifying Compound with UDETA

A comparative oligoamide was prepared by using UDETA in place of the modifying group UDETA-C6 according to the invention. This material was prepared according to a process similar to that shown in example 2, starting from 50 g of Pripol® 1009 (0.175 mol), 24 g of Priamine® 1074 (0.088 mol) and 11.32 g of UDETA (0.088 mol), except that the Priamine® and the UDETA were introduced into the reactor heated to 180° C. before adding the Pripol® 1009.

The product obtained existed, at ambient temperature, in the form of a very viscous liquid not exhibiting any significant mechanical property. Its viscosity was less than 0.3 Pa·s at 180° C.

1. A material comprising linear or branched oligoamides X.Y terminated at more than 90% by number of their ends by one and the same group -M-L₂-CO-L₁-A, where A is an associative group comprising a nitrogenous heterocycle; L₁ is a chemical bond or a spacer arm consisting of a saturated or unsaturated and cyclic or noncyclic hydrocarbon chain optionally interrupted by one or more oxygen and/or nitrogen atoms; L₂ is a saturated or unsaturated and cyclic or noncyclic hydrocarbon chain which includes at least 4 carbon atoms, which is optionally interrupted by one or more oxo groups and which is optionally substituted by one or more —OH groups and/or one or more chlorine atoms; and M is selected from the group consisting of CO, NH and O groups.

2. The material as claimed in claim 1, wherein the material comprises oligomers corresponding to the formula (Ia) below:
X—NH—Ra—NH—[CO—Rb—CO—NH—Ra—NH—]ₐ—X (Ia)
where:
Ra denotes a saturated or unsaturated hydrocarbon chain optionally interrupted by one or more oxygen and/or nitrogen atoms,
Rb denotes a saturated or unsaturated hydrocarbon chain,
a denotes the mean number of units per chain and is greater than 0 and less than or equal to 20,
X denotes an A-L₁-CO-L₂-CO— group
where:
A is an associative group comprising a nitrogenous heterocycle,
L₁ is a chemical bond or a spacer arm consisting of a saturated or unsaturated and cyclic or noncyclic hydrocarbon chain optionally interrupted by one or more nitrogen and/or oxygen atoms, and
L₂ is a saturated or unsaturated and cyclic or noncyclic hydrocarbon chain which includes at least 4 carbon atoms, which is optionally interrupted by one or more oxo groups and which is optionally substituted by one or more —OH groups and/or one or more chlorine atoms.

3. The material as claimed in claim 1, wherein the material comprises oligomers corresponding to the formula:
X′—CO—Rb—CO—[NH—Ra—NH—CO—Rb—CO—]ₐ—X′ (Ib)
where:
Ra denotes a saturated or unsaturated hydrocarbon chain optionally interrupted by one or more oxygen and/or nitrogen atoms,
Rb denotes a saturated or unsaturated hydrocarbon chain,
a denotes the mean number of units per chain and is greater than 0 and less than or equal to 20,
X′ denotes an A-L₁-CO-L₂-M₁- group
where:
A is an associative group comprising a nitrogenous heterocycle,
L₁ is a chemical bond or a spacer arm consisting of a saturated or unsaturated and cyclic or noncyclic hydrocarbon chain optionally interrupted by one or more nitrogen and/or oxygen atoms,
L₂ is a saturated or unsaturated and cyclic or noncyclic hydrocarbon chain which includes at least 4 carbon atoms, which is optionally interrupted by one or more oxo groups and which is optionally substituted by one or more —OH groups and/or one or more chlorine atoms, and
M₁ is an oxygen atom or an NH group.

4. The material as claimed in claim 2, wherein one at least of the Ra and Rb chains does not consist of a linear alkylene chain.

5. The material as claimed in claim 1, wherein the associative group is selected from the group consisting of imidazolidonyl, triazolyl, triazinyl, bis-ureyl and ureido-pyrimidyl groups.

6. The material as claimed in claim 1, wherein the material exhibits at least one of the following properties:
a semicrystalline nature, with a melting temperature (Tₘ) of between 120 and 260° C. and a glass transition temperature (T_{g}) of between −25° C. and 100° C.,
a number-average molecular weight, as measured by GPC, of less than 4500 g/mol,
a melt viscosity of less than 10 Pa·s,
a breaking stress of greater than 1 MPa.

7. A process for the preparation of the material as claimed in claim 1, comprising a stage of polycondensation:
(a) of at least one dicarboxylic acid,
(b) of at least one diamine, and
(c) of at least one modifying compound of formula A-L₁-CO-L₂-W, where A is an associative group comprising a nitrogenous heterocycle; L₁ is a chemical bond or a spacer arm consisting of a saturated or unsaturated and cyclic or noncyclic hydrocarbon chain optionally interrupted by one or more oxygen and/or nitrogen atoms; L₂ is a saturated or unsaturated and cyclic or noncyclic hydrocarbon chain which includes at least 4 carbon atoms, which is optionally interrupted by one or more oxo groups and which is optionally substituted by one or more —OH groups and/or one or more chlorine atoms; and W is a reactive group capable of reacting:
either with the amine functional groups of the diamine, the molar ratio of the acid functional groups of the diacid to the reactive functional groups of the modifying compound ranging from 1 to 8, it being understood that the number of moles of amine functional groups of the diamine is equal to the sum of the number of moles of the abovementioned acid and reactive functional groups,
or with the acid functional groups of the diacid, the molar ratio of the amine functional groups of the diamine to the reactive functional groups of the modifying compound ranging from 1 to 8, it being understood that the number of moles of acid functional groups of the diacid is equal to the sum of the number of moles of the abovementioned amine and reactive functional groups.

8. The process as claimed in claim 7, wherein the dicarboxylic acid is an aromatic diacid or a saturated linear dicarboxylic acid including from 4 to 22 carbon atoms.

9. The process as claimed in claim 7, wherein the diamine is selected from the group consisting of polyetheramines comprising a linear or branched polyether chain, each end of which carries a primary amine group, branched diamines and polyamines, the chain of which comprises a sulfur atom, and dimer diamines resulting from vegetable fatty acids which can be partially or completely hydrogenated.

10. The process as claimed in claim 7, wherein the modifying compound corresponds to the formula (IV): 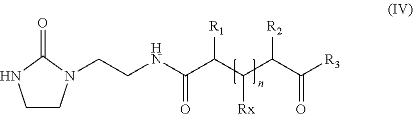
in which R₁, R₂ and each of the Rx groups independently denote a hydrogen atom, an —OH group or a —CH₃ group; n ranges from 2 to 12; and R₃ denotes an —OH group, an —OCH₃ group or a chlorine atom.

11. The process as claimed in claim 10, wherein the modifying compound corresponds to the following formula (V): 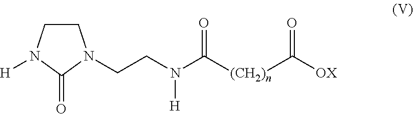
where n is between 4 and 14 and X═H or CH₃.

12. (canceled)

13. A compound of formula: 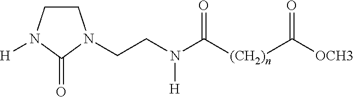
where n ranges from 4 to 14.

14. (canceled)

15. The material as claimed in claim 3, wherein one at least of the Ra and Rb chains does not consist of a linear alkylene chain.

16. A method for manufacturing a product selected from the group consisting of leaktight seals, thermal and acoustic insulators, footwear soles, packagings, coatings, systems for trapping and releasing active principles, vacuum pipes, rheological additives, additives for asphalt, additives for hot-melt adhesives, and matrices of composites, wherein the method comprises using a material as claimed in claim 1 in the manufacturing.

17. The process of claim 7, wherein the modifying compound has formula: 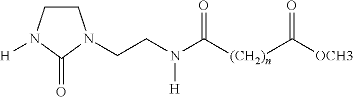
wherein n ranges from 4 to 14.

18. The material as claimed in claim 1, wherein the material exhibits all of the following properties:
a semicrystalline nature, with a melting temperature (Tₘ) of between 120 and 260° C. and a glass transition temperature (T_{g}) of between −25° C. and 100° C.,
a number-average molecular weight, as measured by GPC, of less than 4500 g/mol,
a melt viscosity of less than 10 Pa·s,
a breaking stress of greater than 1 MPa.

19. The material of claim 2, wherein a is between 1 and 3.

20. The material of claim 3, wherein a is between 1 and 3.

21. The material as claimed in claim 3, wherein one at least of the Ra and Rb chains does not consist of a linear alkylene chain.

22. The material as claimed in claim 1, wherein the associative group is an imidazolidonyl group.
